(19) United States
(12) Patent Application Publication
(10) Pub. No.: US 2002/0012630 A1
(43) Pub. Date: Jan. 31, 2002

(54) Methods for testing compounds useful in treating diabetic complications

(76) Inventors: James Nolan, Guilford, CT (US); Cheryl Armstrong, Atlanta, GA (US); John C. Ansel, Atlanta, GA (US)

Correspondence Address: Kevin E. Noonan, McDonnell Boehnen Hulbert & Berghoff, 32nd Floor, 300 S. Wacker Drive, Chicago, IL, 60606, US

(21) Appl. No.: 09/851,743

(22) Filed: May 9, 2001

Related U.S. Application Data
(60) Provisional application No. 60/203,151, filed on May 9, 2000.

(51) Int. Cl. A61K 49/00

(52) U.S. Cl. 424/9.2

(57) ABSTRACT

The present invention provides methods for testing compounds to determine whether such compounds are useful for improving treatment of wounds in animals, particularly diabetic animals, and improving diabetic neuropathy or neurological disorders associated with diabetes. The invention also provides methods for characterizing compounds, particularly aldose reductase inhibitors, that are useful in improving wound healing in diabetic animals or improving diabetic neuropathy or neurological disorders associated with diabetes. In addition, the invention relates to methods for improving wound healing and improves diabetic neuropathy or neurological disorders associated with diabetes in diabetic animals using aldose reductase inhibitors.

This application claims priority of U.S. Provisional Patent Application Ser. No. 60/203,151, filed on May 9, 2000, the disclosure of which is incorporated herein by reference in its entirety.

### BACKGROUND OF THE INVENTION

1. Field of the Invention

This invention relates to methods for testing compounds to determine whether such compounds are useful for improving treatment of diabetic complications in animals with diabetes. Specifically, the invention relates to methods for characterizing compounds, particularly aldose reductase inhibitors, that are useful in improving wound healing in diabetic animals and improving diabetic neuropathy or neurological conditions of diabetes. In addition, the invention relates to compounds and methods for improving wound healing, diabetic neuropathy and neurological conditions of diabetes in diabetic animals using aldose reductase inhibitors.

2. Background of the Invention

Diabetes mellitus (DM) is a disease that adversely affects many organ systems, including the skin. Cutaneous problems associated with DM include loss of skin integrity (diabetic dermopathy and necrobiosis lipoidica), blistering of the skin (bullous dermatosis of diabetes), and ulcerations of the skin (neurotrophic ulcers). In addition, wounds in diabetics, whether resulting from one of the above conditions or from trauma, heal more slowly than wounds in non-diabetics. These slow-healing wounds, which are often located on the lower extremities, can result in significant morbidity. Diabetics also may have peripheral neuropathy in the lower extremities, which exacerbates morbidity caused by slow wound healing, at least because components of the cutaneous neurologic system appear to play a role in wound healing in both humans and mice.

Diabetic patients with severe peripheral neuropathy and cutaneous non-healing wounds have been shown to lack nerve fibers in the epidermis that stain for the peptide gene product (PGP) 9.5 (Kennedy et al., 1996, Neurology 47: 1042-1048). These patients also suffer from markedly reduced numbers of such nerve fibers in the dermis surrounding skin ulcerations, in normal skin adjacent to the ulcers, and on proximal leg skin. In addition, these patients lack nerve fibers in the epidermis and papillary dermis of the skin surrounding the non-healing wound that stain for the calcitonin gene related peptide (CGRP; Gibran et al., 2001, submitted for publication). Lower amounts of CGRP-staining nerve fibers, however, can be detected in the reticular dermis near ulcers, and nearly normal amounts of CGRP staining nerve fibers are found in the proximal leg dermis.

Similar results have been obtained with skin biopsy specimens collected from skin adjacent to chronic wounds in patients suffering from spinal cord injury and loss of neurologic function (Chiu et al., 1996, Joint Meeting of the Wound Healing Society and the European Tissue Repair Society, Boston, Mass.). In this study, patients undergoing rotation flaps to close pressure ulcers in the sacral region were observed to have a marked decrease in epidermal innervation in the skin adjacent to the patients' wounds. While an intermediate decrease in innervation was observed in the skin from the patients' legs (i.e., in areas where the patients lacked normal sensation), normal innervation was observed in the skin of the patients' arms (i.e., in areas where the patients had normal sensation). Such results demonstrate the functional and morphologic reduction of innervation in the skin in the region of non-healing ulcers in both diabetic and spinal cord injury patients with severe neuropathy. These results also suggest that decreased innervation in the skin may contribute to the failure of wound healing in diabetics and spinal cord injury patients.

Diabetic mice have provided a good model system for studying decreased innervation in wound healing. For example, in diabetic mice, a pattern of decreased epidermal innervation for both PGP 9.5 and CGRP-staining nerve fibers has been observed that is very similar to the pattern described above for diabetic human patients (Underwood et al., 1999, Invest. Dermatol. 2: 631). In addition, diabetic mice show impaired cutaneous wound healing. In studies in which wounds in normal mice healed in 20 days, wounds in diabetic mice required 41.2 days to heal (p<0.001).

Substance P (SP) has been shown to improve wound healing in neuro-impaired db/db mice (Gibran et al., 1998, Society of Academic Surgery Meeting, Seattle, Wash.). In the wound healing studies, three doses of SP were added on days 0, 1, and 2 under a Tegederm wound dressing to groups of db/db mice with experimentally induced full-thickness skin wounds. The addition of SP was found to lead to an improved time of healing in db/db mice treated with a dose of 10-1000 nM SP. These results further suggest that cutaneous neurosensory neuropeptides may play a positive role in wound healing. Lack of innervation, and thus loss of neurosensory neuropeptides, may retard wound healing.

The use of aldose reductase inhibitors (ARIs) for the treatment of diabetic complications is well known in the art. Diabetic complications arise from elevated levels of glucose in tissues such as the nerve, kidney, retina, and lens. Glucose enters the polyol pathway and is converted to sorbitol via aldose reductase. Because sorbitol does not easily cross cell membranes, it accumulates inside cells resulting in changes in osmotic pressure, alterations in the redox state of pyridine nucleotides (i.e., increased NADH/NAD⁺ ratio), and depleted intracellular levels of myo-inositol. Sorbitol, fructose, and advanced glycation products of fructose, have been shown to cause covalent modification and inactivation of structural proteins critical for neural function. In addition, it has been suggested that increased aldose reductase activity yields a decrease in nerve myo-inositol, which results in a decrease in Na⁺/K⁺-ATPase activity, which in turn causes decreased nerve function. Increases in aldose reductase activity resulting from hyperglycemia have also been shown to produce nerve ischemia and nerve dysfunction, by competing with nitric oxide synthetase for NADH, causing a decrease in nitric oxide, which in turn results in a decrease in nerve blood flow (Tilton et al., 1993, Diabetes 42:221-32). In addition, the decrease in nitric oxide production during wound healing leads to a decrease in the deposition of wound reparative collagen (Schaffer et al., 1997, Surgery 121:513-19).

These biochemical changes, which have been linked to diabetic complications, can be controlled by inhibitors of aldose reductase. For example, U.S. Patent No. 6,214,991 (to Jones et al.) teaches a class of compounds, i.e., substituted indolealkanoic acids, that react with and inhibit aldose reductase. Aldose reductase inhibitors have been shown to increase the levels of nerve growth factor in the sciatic nerves of diabetic rats and to stimulate the synthesis and secretion of nerve growth factor in rat Schwann cell cultures (Ohi et al., 1998, Exp. Neurol. 15:215-20). In diabetic patients, treatment with aldose reductase inhibitors has also been shown to cause the regeneration of unmyelinated nerve fibers of the sensory nerve.

There remains a need in the art for effective methods for testing compounds, particularly aldose reductase inhibitors, to identify compounds useful for treating wounds in diabetic animals. The development of such methods would have wide application in the medical arts.

### SUMMARY OF THE INVENTION

As used herein, “identify” or “identifying” means determining which compound(s), if any, out of a pool of one or more compounds (“test compounds”) has the desired activity or characteristic.

This invention relates to methods for identifying compounds that improve wound healing in diabetic animals. The invention provides such methods, in one embodiment using experimental animals to identify such compounds and in another embodiment using human subjects to identify compounds useful in improving wound healing in diabetic patients. In a related aspect, the methods rely on the use of human subjects to confirm the activity of compounds identified using in vitro or experimental animal assays. In a specific aspect, the compound is an aldose reductase inhibitor (ARI).

The invention also provides methods for improving wound healing in diabetic animals, most preferably human diabetic patients. These methods of the invention comprise administering to the human an aldose reductase inhibitor identified using the methods of the invention in a dose that is effective in improving wound healing in the animal.

The invention in yet another aspect provides methods for identifying compounds that improve diabetic neuropathy and neurological disorders associated with diabetes, the method comprising assaying wound healing in diabetic animals, most preferably human diabetic patients, in the presence and absence of the compound. In a specific embodiment of this aspect, the compound is an aldose reductase inhibitor.

The invention in yet a further aspect provides methods for improving diabetic neuropathy and neurological disorders associated with diabetes, the method comprising administering to the human, a compound, preferably, an aldose reductase inhibitor, identified using the methods of the invention in a dose that is effective in improving diabetic neuropathy and neurological disorders associated with diabetes in the animal.

The invention is advantageous, inter alia, because it permits the rapid and efficacious screening of compounds, for example, in a clinical setting, particularly aldose reductase inhibitors, using an established animal model to identify compounds such as ARIs that are effective in promoting wound healing, and also enables the effectiveness of these compounds in treating diabetic neuropathy to be tested in human diabetics. The invention also is advantageous for providing, for the first time, methods for identifying compounds effective in improving diabetic neuropathy and neurological disorders associated with diabetes in an animal by identifying compounds that promote wound healing in diabetic animals. This aspect of the invention is related to the heretofore unappreciated relationship between diabetic neuropathy and neurological disorders associated with diabetes and impaired wound healing in such animals. In additional embodiments, the ARIs identified by the invention produce improved cutaneous neurologic functioning in diabetics, particularly in extremities and most preferably in the legs.

Specific preferred embodiments of the present invention will become evident from the following more detailed description of certain preferred embodiments and the claims.

### DESCRIPTION OF THE DRAWINGS

FIG. 1 illustrates the aldose reductase pathway.

FIG. 2 illustrates wound healing at 0, 3, 6, and 23 days following wound induction in normal rats and 9-week streptozotocin-induced diabetic rats.

FIG. 3 illustrates a typical wound contraction curve in an animal wound healing model (Clinical and Experimental Approaches to Dermal and Epidermal Repair: Normal and Chronic Wounds, 301-312 (1991)).

FIGS. 4A-4B illustrate wound healing in normal rats and 9-week streptozotocin-induced diabetic rats.

FIG. 5 illustrates wound healing at 0, 9, and 21 days following wound induction in normal untreated rats and 6-week streptozotocin-induced diabetic rats treated with 3-(4,5,7-triflurobenzothiazol-2-yl)methyl-indole-N-acetic acid.

FIGS. 6A-6B illustrate wound healing in normal rats and 6-week streptozotocin-induced diabetic rats treated with 3-(4,5,7-triflurobenzothiazol-2-yl)methyl-indole-N-acetic acid, and normal untreated rats.

FIG. 7 illustrates blood glucose levels in diabetic rats following induction of diabetes with streptozotocin.

FIG. 8 illustrates blood glucose levels in diabetic rats following induction of diabetes with streptozotocin; 20 mg/dL 3-(4,5,7-triflurobenzothiazol-2-yl)methyl-indole-N-acetic acid was administered to diabetic rats at wound induction.

FIG. 9 illustrates the relationship between blood glucose level and wound healing in streptozotocin-induced diabetic rats.

FIGS. 10A-10B illustrate blood glucose levels in untreated streptozotocininduced diabetic rats (A) and streptozotocin-induced diabetic rats treated with 3(4,5,7-triflurobenzothiazol-2-yl)methyl-indole-N-acetic acid.

### DETAILED DESCRIPTION OF THE PREFERRED EMBODIMENTS

The invention provides methods for identifying compounds that improve wound healing in diabetic animals. In a preferred embodiment of the method of the invention, the compound is an aldose reductase inhibitor (ARI) or a compound suspected of having such activity. Alternatively, the compound is not known to be an ARI but is predicted to have activity in promoting wound healing or diabetic neuropathy. The invention also provides methods for identifying compounds that improve diabetic neuropathy and neurological disorders associated with diabetes in diabetic animals. Also provided are methods for promoting wound healing and improvement in diabetic neuropathy and neurological disorders associated with diabetes using ARIs and, preferably, other compounds identified by the methods provided herein.

As used herein, an “aldose reductase inhibitor” is any compound, most preferably a compound that can be administered to an animal, preferably a human and most preferably a human in need of such treatment including but not limited to a human with diabetes mellitus (DM), wherein the compound inhibits aldose reductase. Examples of aldose reductase inhibitors that may be used in the methods of the present invention include those described in: (a) U.S. Pat. No. 5,700,819: 2-Substituted benzothiazole derivatives useful in the treatment of diabetic complications, (b) U.S. Pat. No. 4,868,301: Processes and intermediates for the preparation of oxophthalazinyl acetic acids having benzothiazole or other heterocyclic side chains, (c) U.S. Pat. No. 5,330,997: 1H-indazole-3-acetic acids as aldose reductase inhibitors, and (d) U.S. Pat. No. 5,236,945: 1H-indazole-3-acetic acids as aldose reductase inhibitors, the disclosures of which are incorporated herein by reference in their entirety.

The methods provided by the invention are useful for identifying compounds that improve wound healing in animals, preferably humans and most preferably humans with diabetes. As used herein, the term “wound healing” is intended to refer to wounds to skin or other external body surfaces, including but not limited to cuts, scrapes, punctures, and burns. In order to “improve” wound healing, said compounds must decrease the time taken for a wound to heal, lessen the pain associated with wound healing, reduce the amount or extent of scar tissue formation produced by the healing process, or promote the more complete healing of the wound.

The methods provided by the invention are also useful for identifying compounds that improve neuropathy, particular neuropathy of the extremities and most particularly neuropathy of the lower extremities and feet, and improve neurological conditions associated with diabetes, preferably human diabetes patients. As used herein, the term “improve neuropathy” and “improve neurological conditions associated with diabetes” is intended to refer to increased sensory nerve function and sensation in the extremities, particularly the lower extremities. Thus, the invention provides in vivo assays for discovering compounds that have activity in treating or improving neuropathy, preferably diabetic neuropathy or other neurological conditions associated with diabetes.

This invention provides assays for wound healing that act as surrogates for improving neuropathy and neurological conditions associated with diabetes. It has been unexpectedly found that compounds, particularly, for example, ARIs, which are effective in promoting wound healing in diabetic animals are also effective in improving neuropathy and neurological conditions associated with diabetes. In other words, improvement in wound healing can be considered a marker for improving or treating neuropathy. Thus, the invention provides assays that are more rapid, more convenient, more easily performed, require less complicated equipment and less skilled operators and that have defined endpoints (wound healing) that are more easily detected and quantitated than the more subjective improvement in neuropathy resulting in greater sensory nerve function and sensation in the extremities.

Preferred methods provided by the invention permit identification of compounds, preferably novel compounds, having ARI activity and provide for the efficient characterization of such compounds as ARIs. These novel compounds, and the methods for identifying and characterizing said novel compounds, comprise, inter alia, related aspects of the invention. Said inventive methods also enable such newly-identified ARIs to be formulated into pharmaceutical compositions for use in an animal, most preferably a human, and methods for administering said novel ARIs to achieve a therapeutic result.

The pharmaceutical compositions of the present invention may be prepared in various forms for administration, including tablets, caplets, pills or dragees, or can be filled in suitable containers, such as capsules, or, in the case of suspensions, filled into bottles. As used herein “pharmaceutically acceptable carrier medium” includes any and all solvents, diluents, or other liquid vehicle; dispersion or suspension aids; surface active agents; preservatives; solid binders; lubricants and the like, as suited to the particular dosage form desired. Various vehicles and carriers used in formulating pharmaceutical compositions and known techniques for the preparation thereof are disclosed in Remington's Pharmaceutical Sciences (A. Osol et al. eds., 15th ed. 1975). Except insofar as any conventional carrier medium is incompatible with the chemical compounds of the present invention, such as by producing any undesirable biological effect or otherwise interacting in a deleterious manner with any other component of the pharmaceutical composition, the use of the carrier medium is contemplated to be within the scope of this invention.

In the pharmaceutical compositions of the present invention, the active agent ay be present in an amount of at least 1% and not more than 95% by weight, based on the total weight of the composition, including carrier medium or auxiliary agents. Preferably, the proportion of active agent varies between 1% to 70% by weight of the composition. Pharmaceutical organic or inorganic solid or liquid carrier media suitable for enteral or parenteral administration can be used to make up the composition. Gelatin, lactose, starch, magnesium, stearate, talc, vegetable and animal fats and oils, gum polyalkylene glycol, or other known excipients or diluents for medicaments may all be suitable as carrier media.

The pharmaceutical compositions of the present invention may be administered using any amount and any route of administration effective for increasing the therapeutic efficacy of drugs. Thus the expression “therapeutically effective amount,” as used herein, refers to a sufficient amount of the chemosensitizing agent to provide the desired effect against target cells. The exact amount required will vary from subject to subject, depending on the species, age, and general condition of the subject; the particular chemosensitizing agent; its mode of administration; and the like.

The pharmaceutical compounds of the present invention are preferably formulated in dosage unit form for ease of administration and uniformity of dosage. “Dosage unit form,” as used herein, refers to a physically discrete unit of therapeutic agent appropriate for the animal to be treated. Each dosage should contain the quantity of active material calculated to produce the desired therapeutic effect either as such, or in association with the selected pharmaceutical carrier medium. Typically, the pharmaceutical composition will be administered in dosage units containing from about 0.1 mg to about 10,000 mg of the agent, with a range of about 1 mg to about 1000 mg being preferred.

The pharmaceutical compositions of the present invention may be administered orally or paternally, such as by intramuscular injection, intraperitoneal injection, BY intravenous infusion, or most preferably, topically. The pharmaceutical compositions may be administered orally or parenterally at dosage levels of about 0.1 to about 1000 mg/kg, and preferably from about 1 to body weight per day, one or more times a day, to obtain the desired therapeutic effect.

Although the pharmaceutical compositions of the present invention can be administered to any subject that can benefit from the therapeutic effects of the compositions, the compositions are intended particularly for the treatment of diseases in humans.

The pharmaceutical compositions of the present invention will typically be administered from 1 to 4 times a day, so as to deliver the daily dosage as described herein. Alternatively, dosages within these ranges can be administered by constant infusion over an extended period of time, usually 1 to 96 hours, until the desired therapeutic benefits have been obtained. However, the exact regimen for administration of the chemical compounds and pharmaceutical compositions described herein will necessarily be dependent on the needs of the animal being treated, the type of treatments being administered, and the judgment of the attending physician.

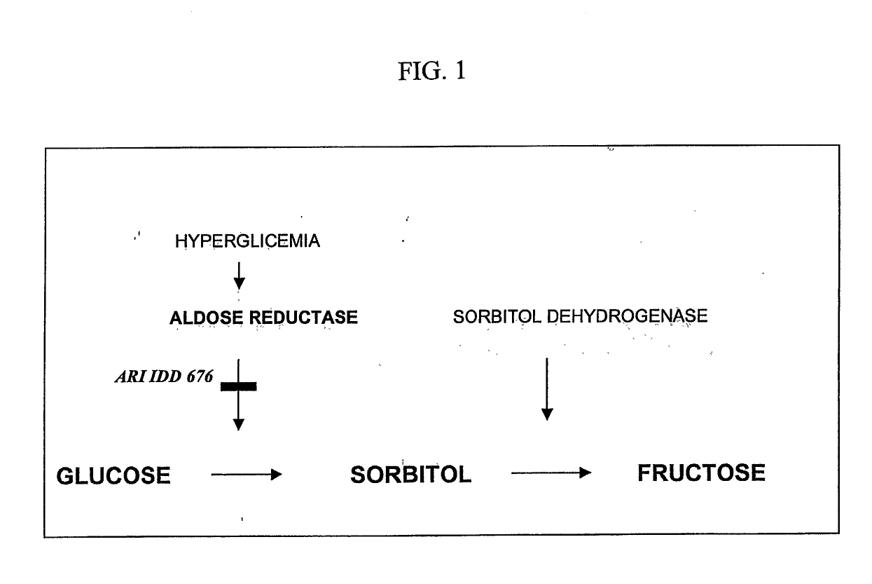

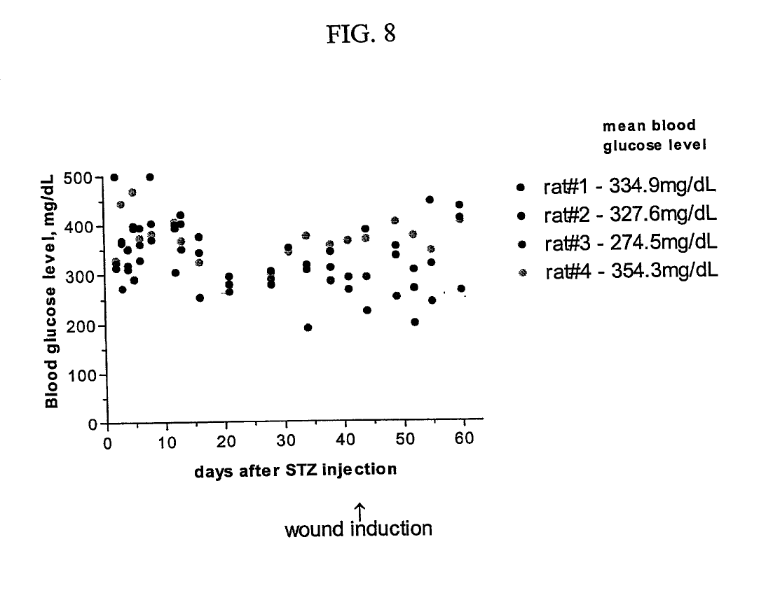

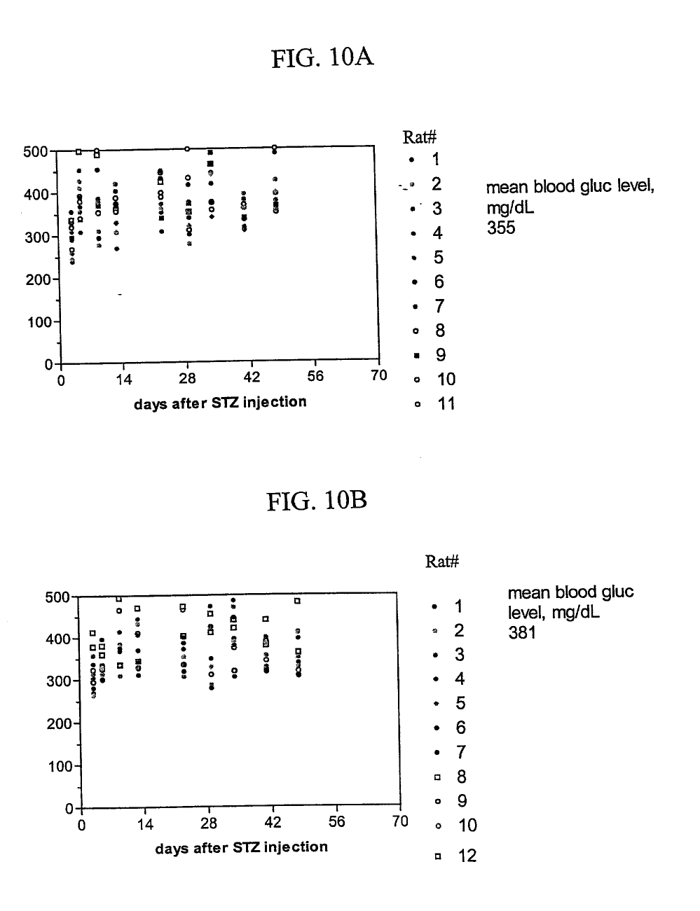

Preferred embodiments of the present invention and its advantages are best understood by referring to FIGS. 1-10 and Examples 1-2. The Examples, which follow, are illustrative of specific embodiments of the invention, and various uses thereof. They are set forth for explanatory purposes only, and are not to be taken as limiting the invention.

### EXAMPLE 1

### Treatment of Wounds in Diabetic Rats

The ability of aldose reductase inhibitors to enhance wound healing in experimental wounds of diabetic rats was examined as follows.

Diabetes was experimentally induced in rats by intraperitoneal injection of 65 mg/kg streptozotocin 24 hours after fasting, which destroys the pancreatic islets cells preventing the production of endogenous insulin. The blood glucose levels of streptozotocin-induced diabetic rats was maintained at between 300-350 mg/dL with subcutaneous injection of insulin.

Experimental wounds, having a diameter of 1.5 cm² and including the panniculus carnosus muscle, were made on the backs of diabetic rats. Wounds were serially photographed with a digital camera at the time of wounding, and then again every three days after wound induction until the time of closure, and the area of the wound measured (and expressed as cm²). Wound closure was defined as complete epithelialization. The degree of wound contraction is calculated as: {(size of wound at day 0−size of wound at day X)/size of wound at day 0)}×100.

Diabetic rats were administered 20 mg/kg of the aldose reductase inhibitor 3(4,5,7-triflurobenzothiazol-2-yl)methyl-indole-N-acetic acid at either the time of diabetes induction (simulating treatment prior to diabetic complications) or 6 weeks after diabetes induction (simulating treatment at the time of diabetic complications).

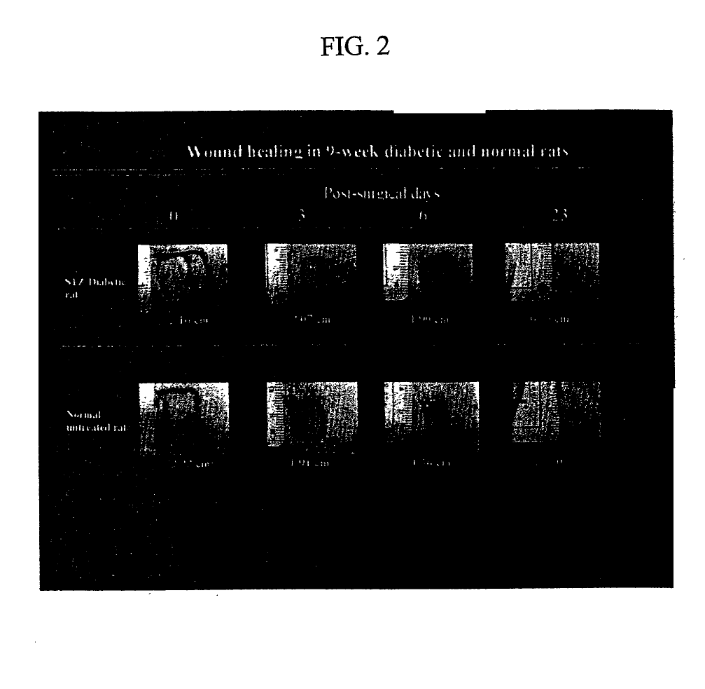

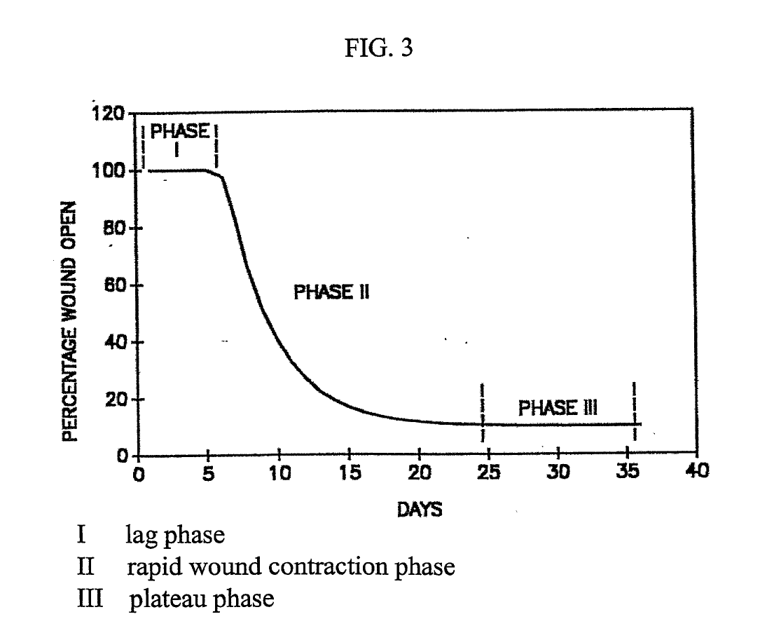

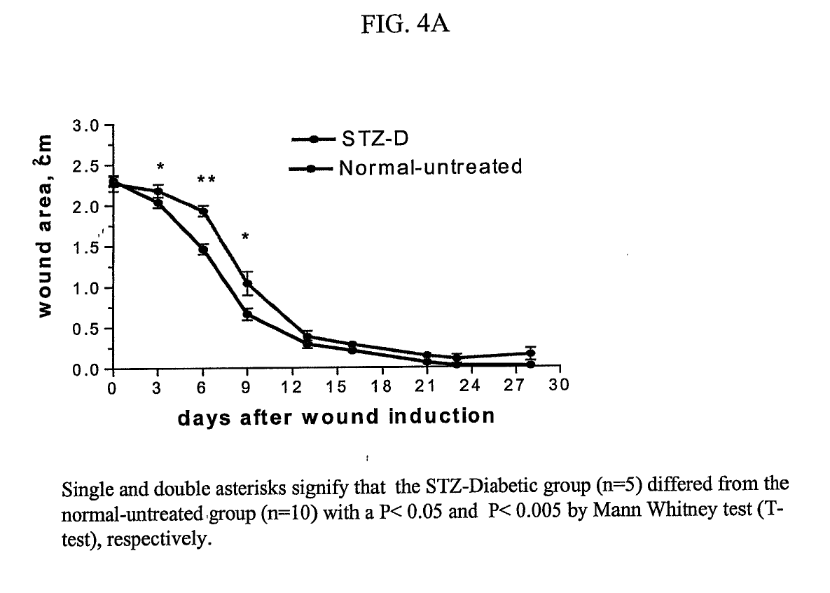

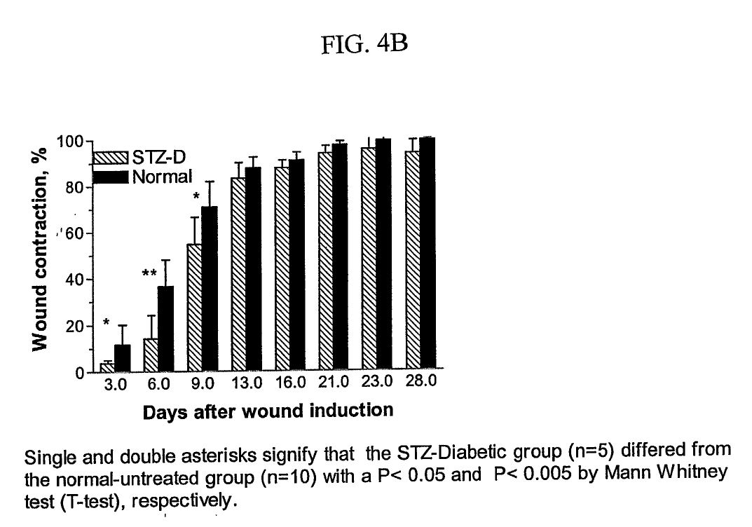

Digital photographs of wound healing in normal rats and 9-week streptozotocin-induced diabetic rats are shown in FIG. 2. These images were used to calculate wound size and the degree of wound contraction. A typical wound contraction curve in an animal wound healing model is shown in FIG. 3. The results of wound size and wound contraction calculations for the images shown in FIG. 2 are indicated in FIGS. 4A-4B. As is evident from these figures, the wounds in streptozotocin-induced diabetic rats are slower to heal during the rapid wound contraction phase than are wounds in normal rats.

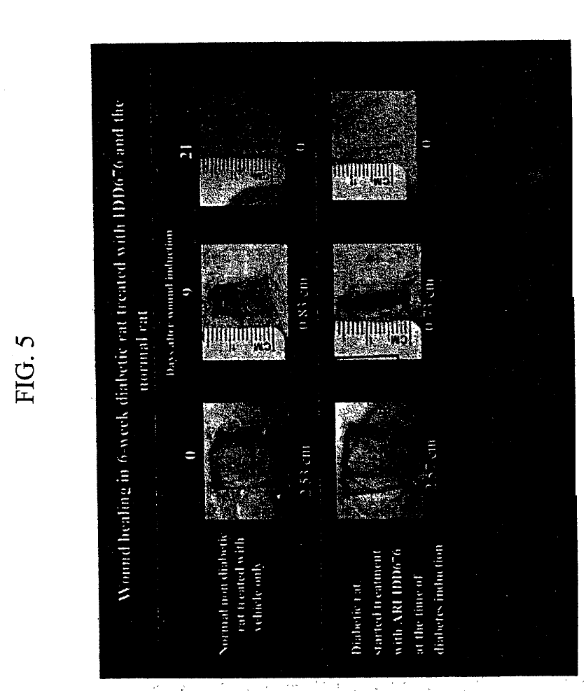

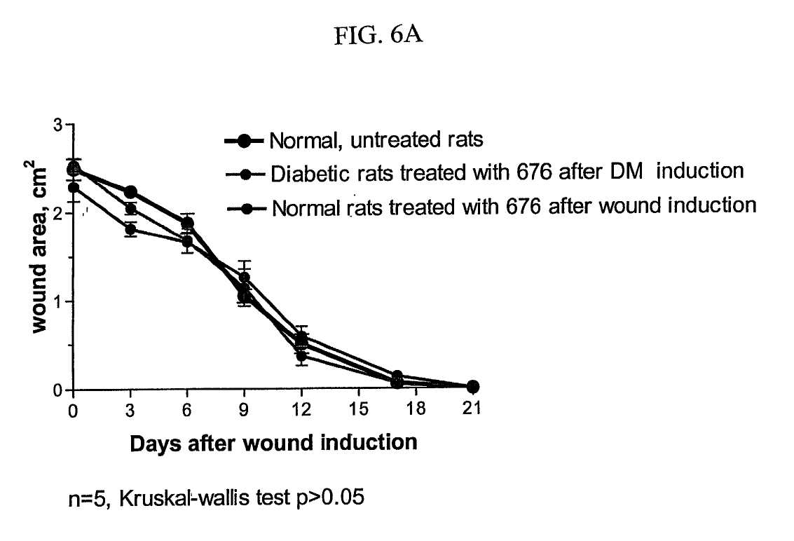

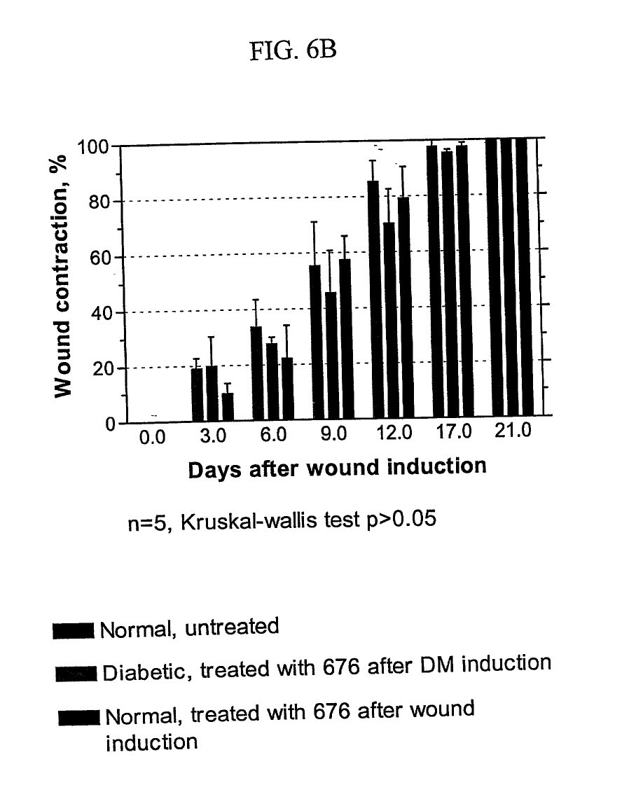

Digital photographs of wound healing in normal untreated rats and 6-week streptozotocin-induced diabetic rats treated with 3-(4,5,7-triflurobenzothiazol-2-yl)methyl-indole-N-acetic acid is shown in FIG. 5. The results of wound size and wound contraction calculations for the images shown in FIG. 5 are indicated in FIGS. 6A-6B. As is evident from these Figures, the wounds in treated streptozotocin-induced diabetic rats healed at the same rate as the wounds in normal rats.

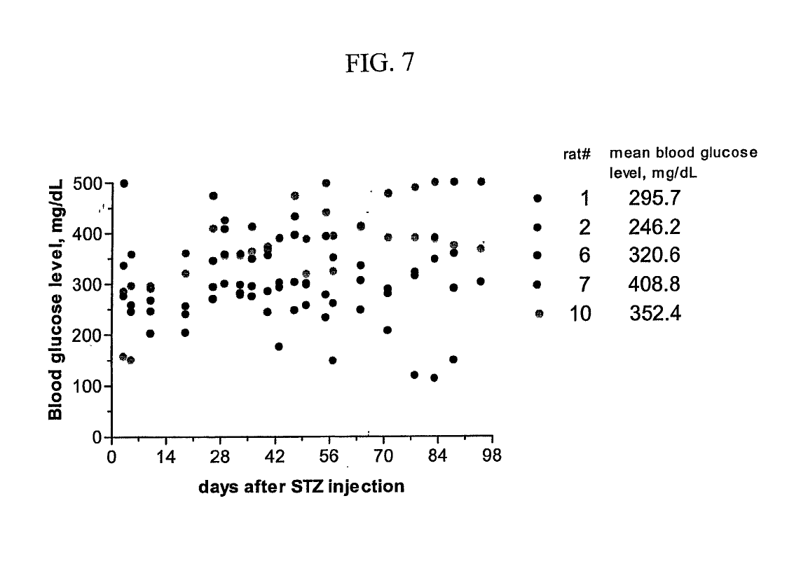

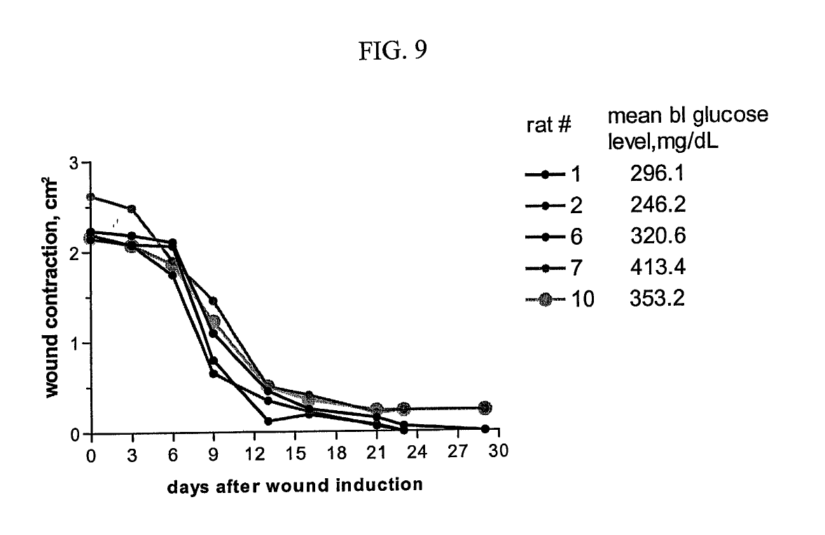

The blood glucose levels in untreated streptozotocin-induced diabetic rats and streptozotocin-induced diabetic rats treated with 3-(4,5,7-triflurobenzothiazol-2-yl)methyl-indole-N-acetic acid are shown in FIGS. 7-8 and 10A-10B. The relationship between blood glucose level and wound healing in streptozotocin-induced diabetic rats is shown in FIG. 9.

These data indicate that the wound healing methods disclosed herein can qualitative and quantitative detect the extent, kinetics and time-course of wound healing using a known ARI. These methods can thus be used to evaluate novel compounds as ARIs and to characterize such compounds with regard to their effect on wound healing in animals.

### EXAMPLE 2

### Impairment of Wound Healing in Diabetic Human Patients

The impairment of wound healing in human diabetic patients, and the correlation between wound healing and peripheral neuropathy in diabetics, is examined in diabetic human patients as follows. Diabetic human patients, meeting specific eligibility criteria, are selected as test subjects. Suitable test subjects will have clinically diagnosed diabetes mellitus (of any stage); be between 40-60 years of age; male or female; ambulatory (with or without assistance); and have no significant abnormalities in serum sodium, potassium, chloride, carbon dioxide, or magnesium. Age-matched non-diabetics, meeting specific eligibility criteria, are selected as control subjects. Suitable control subjects will exhibit no symptoms of diabetes or clinically apparent signs of neurologic disease; be between 40-60 years of age; male or female; ambulatory (with or without assistance); and have no significant abnormalities in serum sodium, potassium, chloride, carbon dioxide, or magnesium. Criteria for disqualifying subjects from the study include current treatment with any investigational drug, current treatment with coumadin or related anticoagulants, history of neurologic disease other than diabetic neuropathy, current ulceration of the lower extremities, history of spontaneous cutaneous ulceration, history of a hypercoagulation disease, electrolyte abnormalities, or significant dermatologic disease affecting the lower extremities that would interfere with wound healing or hinder the evaluation of wound healing.

Non-invasive electrophysiology is used to classify test subjects as Stage I, Stage II, or Stage III diabetic neuropathy. Approximately 45 patients will enroll in the study, with at least ten test subjects with each of Stage I, Stage II, or Stage III neuropathy and at least fifteen control subjects with no detectable neuropathy are selected. For non-invasive electrophysiology procedures, surface stimulating and recording techniques are used. Skin temperature is controlled so that the lower leg temperature is maintained at a minimum of 31.0° C. and the upper limb temperature at a minimum of 32.0° C. for the duration of the testing. The temperature is recorded at the beginning and end of the nerve conduction study. To ensure proper electrode function, the skin is cleaned with alcohol or acetone, and a conducting medium (paste or jelly) applied between the electrode and skin. A ground electrode is placed between the stimulating and recording electrodes. An electrical stimulus with a duration of 0.05 to 0.2 ms is applied to the skin, and the intensity of stimulation increased in small steps until a maximal evoked response is obtained. The stimulus intensity is the increased by 20 to 30% to provide a supramaximal stimulus and corresponding response, and the stimulation rate is 1/sec. Sensory responses are averaged with 5 to 20 responses to eliminate baseline noise and improve accuracy. Motor latencies are measured from the onset of stimulation to the onset of the initial negative peak and are recorded to the nearest 0.1 ms by the internal cursor. The sensitivity of the equipment is set to 500 μV/div to measure the onset latency. Sweep speeds are set at 2 or 3 ms/div for motor latency measurements. Sensory latencies are measured from the onset of stimulation to the positive peak, if present; or to the onset of the initial negative peak, and are recorded to the nearest 0.1 ms with the internal cursor. Sweep speeds are set at 2 ms/div for sensory latency measurements. Amplitudes are measured from the baseline to the negative peak and are recorded to the nearest 0.1 mV for motor responses and the nearest 0.1 μV for sensory responses. The preceding baseline are flat, and without significant stimulus artifact. In motor and sensory conduction studies, the sensitivity for amplitude measures is set to show the entire curve.

In addition, sural nerve conduction and peroneal nerve motor conduction studies are done at the beginning of the study. In this procedure, the active recording electrode (cathode) is placed posterior to the lateral malleolus. The reference electrode (anode) is placed 3.0 cm distal to the active electrode. The ground electrode is positioned between the active electrode and the stimulator. The stimulator cathode is applied approximately 14.0 cm proximal to the active electrode in the lower calf. Stimulation is carried out to achieve a supramaximal response that is then averaged (5 to 20 responses). The cursors for latency and amplitude are set, and the distances recorded. The temperatures at beginning and end of the study are recorded.

For peroneal nerve motor conduction studies, done in the dominant leg at the beginning of the study, the active recording electrode is placed over the endplate region of the extensor digitorum brevis muscle. The reference electrode is placed at the base of the fifth toe. The ground is placed between the recording and stimulating electrodes. The stimulator cathode will be placed over the peroneal nerve at the ankle 9.0 cm proximal to the recording electrode. Stimulation is carried out to achieve a supramaximal response (M-wave). The stimulator cathode is then placed over the peroneal nerve at the knee just distal and lateral to the head of the fibula. Supramaximal stimulation achieves the maximal M-wave from the knee. The cursors for latency and amplitude are set, and the distances recorded. The stimulator is then applied to the ankle again, but the cathode placed proximally, and at least 10 supramaximal stimuli applied. The sweep speed will be set to record F wave latencies, and the minimal, reproducible F wave latency will be measured. The temperatures at beginning and end of the study are recorded.

Experimental wounds are induced on the mid- or lower legs of test and control subjects using a punch biopsy procedure. Prior to biopsy, the skin is prepared with alcohol and anesthetized with infiltration of 0.2-0.3 mL 1% lidocaine with epinephrine. A 5 mm disposable punch biopsy tool is used to induce a standard wound six inches below the patella on the medial lower right leg to a depth of the hub of the punch tool. Hemostasis is obtained by the application of pressure to the wound, the wound is covered with ultrathin Duoderm as a dressing, and the dressing is left in place until the subject returns for follow-up examination. The wound is not sutured. Tissue samples removed in the punch biopsies are divided. Half of each tissue sample is embedded in OCT freezing media (Sakura Tissue Tek, Torrance, Calif.), snap-frozen, and stored at −20° C. The other half of each sample is fixed in 10% neutral buffered formalin and stored at room temperature.

Wounds are serially photographed with a digital camera at the time of wounding, and then again at 3, 7, 9, 11, 14, 16, 18, and 21 days after wound induction (or until the time of closure, if sooner), and the area of the wound measured (and expressed as cm²). Most wounds in the test subjects are expected to close by day 21, while wounds in control subjects are expected to close sooner. The degree of wound contraction is calculated as: {(size of wound at day 0—size of wound at day X)/size of wound at day 0)}×100. Each test group of individuals includes at least one experimental and one “normal” control who are treated with 3-(4,5,7triflurobenzothiazol-2-yl)methyl-indole-N-acetic acid as a positive control for wound healing. After the last photographic examination is performed, the wound is surgically revisited and sutured to ensure the most complete and cosmetically appropriate degree of wound healing in all subjects.

This assay is used to determine the ability of test to enhance wound healing in experimental wounds of diabetic human patients is examined using procedures described herein.

It should be understood that the foregoing disclosure emphasizes certain specific embodiments of the invention and that all modifications or alternatives equivalent thereto are within the spirit and scope of the invention as set forth in the appended claims.

What we claim is:

1. A method for identifying a compound that improves treatment of wounds to skin or another external body surfaces in an animal, the method comprising:
a) producing a wound in the skin or another external body surface of an animal;
b) permitting the wound to heal in the absence of a test compound, or treating the wound with a test compound;
c) comparing wound healing in the presence of the compound to wound healing in the presence of the compound; and
d) identifying a compound that improves treatment of wounds to skin or another external body surfaces in an animal if the wound heals more rapidly, more completely or less painfully in the presence of the compound than in the absence of the compound.

2. The method of claim 1, further comprising:
e) comparing wound healing in the presence of the test compound with wound healing in the presence of an aldose reductase inhibitor, wherein the test compound is identified as a compound that improves treatment of wounds to skin or another external body surfaces in an animal if the wound heals at least as rapidly, completely or less painfully in the presence of the compound as in the presence of the aldose reductase inhibitor.

3. The method of claim 1, wherein the animal is a mammal.

4. The method of claim 1, wherein the animal is a human.

5. The method of claim 1, wherein the human is a diabetic.

6. The method of claim 1, wherein the wound is to skin on an animal.

7. The method of claim 1, wherein the wound is produced by punch biopsy.

8. A method for treating wounds in a diabetic animal, comprising administering to the animal a compound that improves treatment of wounds to skin or another external body surfaces in an animal in an amount effective to improve wound healing in the animal, wherein the compound is identified according to the method of claim 1.

9. The method of claim 8, wherein the animal is a mammal.

10. The method of claim 8, wherein the animal is a human.

11. The method of claim 8, wherein the human is a diabetic.

12. The method of claim 1, wherein the test compound is an aldose reductase inhibitor.

13. A method for identifying a compound that improves diabetic neuropathy or neurological disorders associated with diabetes, the method comprising:
a) producing a wound in the skin or another external body surface of an animal;
b) permitting the wound to heal in the absence of a test compound, or treating the wound with a test compound;
c) comparing wound healing in the presence of the compound to wound healing in the presence of the compound; and
d) identifying a compound that improves diabetic neuropathy or neurological disorders associated with diabetes if the wound heals more rapidly, more completely or less painfully in the presence of the compound than in the absence of the compound.

14. The method of claim 13, further comprising:
e) comparing wound healing in the presence of the test compound with wound healing in the presence of an aldose reductase inhibitor, wherein the test compound is identified as a compound that improves diabetic neuropathy or neurological disorders associated with diabetes if the wound heals at least as rapidly, completely or less painfully in the presence of the compound as in the presence of the aldose reductase inhibitor.

15. The method of claim 13, wherein the animal is a mammal.

16. The method of claim 13, wherein the animal is a human.

17. The method of claim 13, wherein the human is a diabetic.

18. The method of claim 13, wherein the wound is to skin on an animal.

19. The method of claim 13, wherein the wound is produced by punch biopsy.

20. A method for treating diabetic neuropathy or neurological disorders associated with diabetes in a diabetic animal, comprising administering to the animal compound that improves treatment of wounds to skin or another external body surfaces in an animal in an amount effective to improve diabetic neuropathy or neurological disorders associated with diabetes in the animal, wherein the compound is identified according to the method of claim 13.

21. The method of claim 20, wherein the animal is a mammal.

22. The method of claim 20, wherein the animal is a human.

23. The method of claim 20, wherein the human is a diabetic.

24. The method of claim 13, wherein the test compound is an aldose reductase inhibitor.

25. The method of claim 2, wherein the animal is a mammal.

26. The method of claim 2, wherein the animal is a human.

27. The method of claim 2, wherein the human is a diabetic.

28. The method of claim 2, wherein the wound is to skin on an animal.

29. The method of claim 2, wherein the wound is produced by punch biopsy.

30. The method of claim 14, wherein the animal is a mammal.

31. The method of claim 14, wherein the animal is a human.

32. The method of claim 14, wherein the human is a diabetic.

33. The method of claim 14, wherein the wound is to skin on an animal.

34. The method of claim 14, wherein the wound is produced by punch biopsy.
